(19) United States
(12) Patent Application Publication
(10) Pub. No.: US 2020/0253791 A1
(43) Pub. Date: Aug. 13, 2020

(54) FEMININE SANITARY PRODUCT WITH INDICATOR

(71) Applicant: Sustainable Ethical Enterprises Limited, Essex (GB)

(72) Inventors: Gale BOWMAN, Chicago, IL (US); Blanca LESMES, Austin, TX (US)

(73) Assignee: Sustainable Ethical Enterprises Limited, Essex (GB)

(21) Appl. No.: 16/859,434

(22) Filed: Apr. 27, 2020

Related U.S. Application Data
(63) Continuation of application No. PCT/GB2018/053114, filed on Oct. 26, 2018.
(60) Provisional application No. 62/577,626, filed on Oct. 26, 2017.

(51) Int. Cl. A61F 13/42; A61F 13/34

(52) U.S. Cl. CPC A61F 13/42; A61F 13/34; A61F 2013/421; A61F 2013/427; A61F 2013/422; A61F 2013/429

(57) ABSTRACT

A feminine sanitary product with an end-of-use indicator for signaling to the user that the product must be replaced. Embodiments are comprised of all-natural materials that promote user safety and reduction in environmental waste. Embodiments include a tampon comprising an absorbent pledget, waterproofed removal string and natural indicator adapted to provide indicate when predetermined limits on saturation or exposure time are met. Natural visual indicator embodiments rely on a color change in the removal string. Natural sensory indicator embodiments rely on a cooling sensation to indicate end of use.

### CROSS-REFERENCE TO RELATED APPLICATIONS

This application is a continuation of International Application No. PCT/GB2018/053114, filed Oct. 26, 2018, which claims priority to U.S Provisional Application No. 62/577,626, filed on October 26, 2017, the entire contents of each of which are hereby incorporated by reference in its entirety.

### TECHNICAL FIELD

The present invention generally relates to a feminine sanitary product comprising an absorbent pledget, removal string, and indicator. More specifically, the invention relates to a tampon with an indicator (preferably made of all-natural material) that signals when the tampon should be changed.

### BACKGROUND

While it is well known that women must change tampons frequently, few if any, existing tampon products indicate when it is time to do so. For example, a significant part of puberty for many girls is learning how to properly use a tampon. A young girl requires guidance in using a tampon to avoid the risk of leakage and the danger of developing Toxic Shock Syndrome (TSS), which is a serious and potentially life-threatening infection that may occur when a tampon is left in for too long. Unfortunately, an adolescent girl newly experiencing her menstrual cycle will not always have a more experienced female in her company that could help teach her the proper procedure for using and changing a tampon or appropriate time for doing so. Thus, there exists a need for a product that helps the user identify when a tampon must be changed.

Tampons have a steep learning curve for safety, efficacy, and comfort associated with proper use. An end-of-use indicator would provide peace of mind for younger or inexperienced women by subtly introducing direction as to when the user should change the tampon.

Indicators benefit the user by reducing the risk of embarrassing leaks due to an oversaturated tampon, the risk of infection due to prolonged of the tampon, and the risk of damage to the vaginal wall due to discomfort following multiple unnecessary changes of the product. Moreover, indicators can provide a financial benefit for the user by minimizing wasteful use.

Tampons can be made from both synthetic and natural material. Some women prefer natural materials, such as certified organic cotton, over synthetic materials to avoid possible exposure to dioxins, pesticides, and other irritants. Irritants can increase the likelihood of misuse and introduce risks of infection, malodor, discomfort and damage to the vaginal wall.

Additionally, the biodegradable characteristic of natural materials provides a long-term benefit of a reduction in greenhouse gases caused by landfills. This is of particular significance given that disposable feminine hygiene products produce an estimated 300 pounds of waste in a woman's lifetime.

Tampon indicators known in the art typically rely on the use of synthetic material (such as polyester, polypropylene, and nylon) or lack the benefits of the other features disclosed herein. Such known examples include those disclosed in U.S. Pat. No. 3,794,024, US Pub. No. 20150217019 and U.S. Pat. No. 8,247,638, all incorporated by reference herein.

U.S. Pat. No. 3,794,024 refers to a string indicator made of cotton. While cotton may be useful as an indicator, indicators made of cotton alone are prone to expansion and leakage. U.S. Pat. No. 3,794,024 also suggests use of polyester fibers in a string indicator, however use of polyester in that capacity is undesirable for those seeking a natural alternative.

U.S. Pat. No. 8,247,638 refers to different types of cotton or cellulose-based indicators adapted to shrink in the presence of moisture. These types of indicators, however, are less desirable because changing the length of the withdrawal string during use may result in a string length that is too short to properly remove the tampon for some users.

US Pub No. 20150217019 refers to a time-lapse indicator that provides an indication after a predetermined period of time. While knowing if a certain amount of time has passed may be useful under some circumstances, it is also desirable to receive an indication that a particular saturation level has been reached.

### BRIEF SUMMARY

One aspect of the invention is a feminine hygiene product comprised of natural materials. The all-natural tampon comprises a pledget, waterproof removal string, and an end-of-use indicator.

In another aspect of the invention, the all-natural pledget has an absorbent core and an outer cover. The absorbent core and outer cover can include, for example, cotton, hemp, linen, or any combination thereof, or other natural material.

In another aspect of the invention, the all-natural removal string is waterproofed by a wax outer layer, which may include naturally occurring waxes, such as but not limited to, beeswax, soy wax, cottonseed wax, palm wax or any combination thereof.

In another aspect of the invention, the end-of-use indicator uses a water-soluble dye that runs through the length of the waterproofed tampon string. After substantial exposure to menstrual fluids, the dye will cause a color change to permeate through the tampon string indicating that the tampon has reached its saturation point and must be replaced. The waterproofing of the string prevents inadvertent color changes that could result from outside factors affecting the tampon's indicator, such as urination or swimming.

In another aspect of the invention, the end-of-use indicator is a thermochromic dye that runs through the length of the waterproofed tampon string. The thermochromic dye causes a color change to permeate through the tampon string based on a time-sensitive exposure to the user's body heat. The Food and Drug Administration (FDA) recommends that a tampon is worn no longer than eight hours, therefore the dye should have a reaction time in accordance with this recommendation.

In yet another aspect of the invention, the end-of-use indicator is a potential of Hydrogen (pH) solution applied to the waterproofed tampon string. A color change would be observed on the tampon string once the pH reaches 7, indicating the presence of menstrual blood. The change results from the increase in a decrease of acidity in the vaginal environment, which typically has a pH range of 4.0-4.5, due to the presence of blood.

In yet another aspect of the invention, the end-of-use indicator produces a cooling sensation that is activated by the moisture from the menstrual fluid absorbed by the tampon. The indicator is contained between the inner layer of the pledget and protected from premature exposure to moisture by means of a permeable waterproofed layer either located on the outside of the pledget or as an inner layer within the pledget.

In yet another aspect of the invention, the all-natural tampon is packaged in combination with a panty liner.

### BRIEF DESCRIPTION OF THE DRAWINGS

A detailed description of embodiments of the invention is provided below, by way of example only, with reference to the accompanying drawings, in which:

FIG. 1 is a side view of an embodiment of the feminine sanitary product and its applicator;

FIG. 2 is a side view of an embodiment incorporating a dye pellet; and,

FIG. 3 is a side view of exemplary configurations of the combination of the removal string and menstrual fluid indicator;

FIG. 4 is a cross-sectional view of an embodiment that utilizes a cooling sensation indicator.

FIG. 5 is a birds-eye view of a combined tampon and panty liner packaging configuration.

### DETAILED DESCRIPTION OF EMBODIMENTS

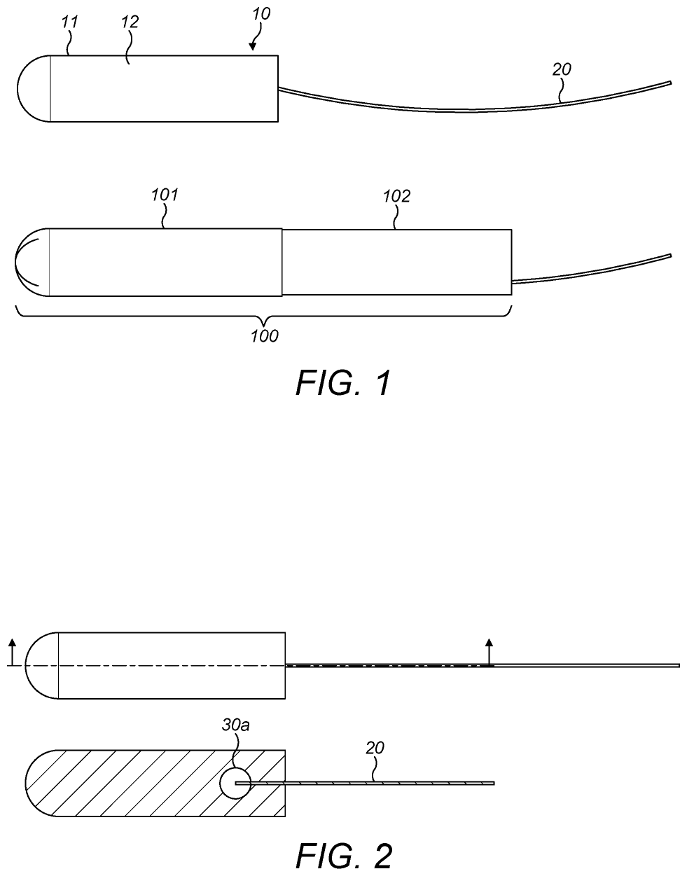

FIG. 1 shows an exemplary embodiment of the feminine sanitary product, generally comprising an absorbent pledget 10, removal string 20, and indicator feature 30. Exemplary embodiments of the indicator feature of the present invention may include a pellet 30a and/or indicator threads 30b. The pledget 10 may be comprised of an outer cover 11 that is immediately exposed to the menstrual fluid and absorbent core 12.

Continuing with FIG. 1, the tampon product may be disposed in an all-natural applicator 100 comprised of a cylinder 101 and a piston 102 made of cardboard or a similar non-synthetic material. The piston 102 fits inside the cylinder 101 and expels the tampon from the applicator 100 in order to commence use.

All aspects of product and applicator may be comprised of all-natural material. While use of the present invention with all-natural materials is preferred, combining it with use of certain synthetic materials (including those manufactured through environmentally friendly and/or sustainable means) may also be possible.

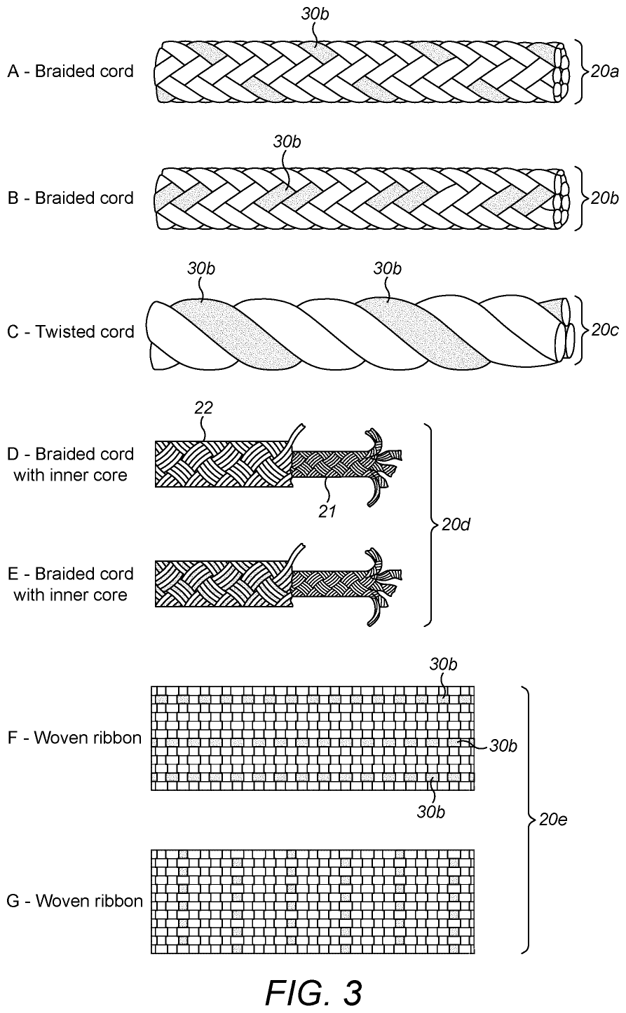

The removal string 20 is integrated with the absorbent core 12, and may be configured to incorporate a moisture activated indicator as shown in FIGS. 2 and 3 and as described in more detail below.

The removal string 20 containing the dye indicator may be waterproofed with a clear material. Possible materials can include, for example, a naturally occurring wax such as beeswax, soy wax, cottonseed wax, palm wax or any combination thereof. The waterproofing also prevents the dye from diffusing out of the removal string 20 and thereby provides an additional protection to the user from inadvertent contact with the colorant. The prolonged exposure of the dye to the menstrual fluid being absorbed by the pledget 10 results in an observable color change.

Referring to FIG. 3, the removal string 20 could be a single layer such as 20a, 20b, and 20c with indicator threads 30b incorporated therein. The indicator threads 30b are depicted by the darker shaded areas in the embodiments of FIG. 3. Possible materials or fibers used for the indicator thread include cotton, linen, or hemp, with at least one of the water-soluble, thermochromic, or pH dyes applied to the thread as the indicator.

Alternatively, the removal string 20d could have an inner core 21 and outer core 22. The inner core 21 may contain indicator threads 30b surrounded by outer core 22 of natural materials, the entirety of which is waterproofed through application of a naturally occurring wax or the like. Another configuration of the removal string 20 could include a waterproofed woven ribbon, such as 20e where indicator threads 30b are intertwined with the removal string 20 threads. Exemplary removal string 20 embodiments could be in the form of cotton string, cord, twine, ribbon, or tape.

Referring to FIG. 3, the braided cord embodiments identified as 20a, 20b and 20c include multiple threads that are braided or twisted together, where certain of the braided or twisted threads are also the indicator threads 30b adapted to alert the user of tampon saturation. The embodiments of 20a and 20b use a cord form where the indicator threads 30b are braided into the cord in several of many possible configurations. The embodiment of 20c uses a twine form with at least one indicator thread incorporated.

The embodiments identified as 20d include an inner core comprised primarily of indicator threads 30b, which, when activated, distribute the dye through outer braided layer, which may also be comprised by at least one indicator thread to further cause the observable color change. The embodiments identified as 20e are in the form of a ribbon or tape, where the indicator threads 30b are woven or knit into several of many possible configurations.

Moisture-Based Indicator: Operation of one exemplary moisture-based indicator occurs as follows. Prior to use the string and pledget are dry at 0% saturation, where the string includes moisture activated dye introduced during manufacture. In one embodiment, the removal string 20 could be predominantly white and the natural water-soluble dye could be applied to at least one thread incorporated into the removal string 20. Exemplary moisture activated (water-soluble) dye compositions include, for example: spirulina extract, annato extract, beta carotene, potassium sodium copper chlorophyllin or similar or related compositions. While in use, the pledget 10 increases in saturation as it is exposed to menstrual fluid. At approximately 60-80% saturation level, dye already present among the fibers of the removal string 20 begins to produce a color change.

Certain embodiments of the present invention use a pellet 30a contained within the pledget, where moisture activated dye is contained within the pellet. Absorbent threads of the string communicate with the pellet through a wicking action, so that prolonged exposure of the dye within the pellet to the menstrual fluid being absorbed by the pledget 10 results in an observable color change of the removal string. The color travels down the string as the pellet 30a and moisture activate dye contained therein absorbs more fluid. The greater the level of saturation, the farther along the string the color travels. A perforated barrier or coating around the pellet may be used to allow moisture to enter, while preventing the dye from diffusing through the absorbent pledget.

pH-Based Indicator: In a pH-based indicator embodiment, the removal string 20 incorporates at least one thread with a pH indicator. The pH indicator is assembled in a manner such that the one end of the removal string is exposed to the absorbent core of the pledget 10 such that color change resulting from the indicator's activation through the moisture in the pledget 10 is then wicked through the removal string to the exposed end. Communication between the pledget and removal string is possible because the moisture wicks through the pledget into the exposed end of the removal string, activating the pH indicator.

The pH indicator must function in the acid-to-neutral range, such that it can capture the pH change from healthy vaginal pH range of 3.8-4.5 to blood pH range of 7.35-7.5. Possible natural pH indicators include fruit-based materials such as blueberries, cherries, grapes, plums, apple skins, and turnips in addition to flower-based materials such as geraniums, roses, tulips, and pansies.

Operation of an exemplary pH based indicator occurs as follows. Prior to use the indicator threads of the removal string are set to a specific color, as determined by the natural pH indicator used. While in use, the pledget 10 absorbs menstrual fluid, and moisture is wicked through the removal string. As the removal string is wetted by menstrual fluid, which is approximately pH 7.35-7.5. the color of the indicator threads changes. The color change travels down the string as the tampon absorbs more fluid. The farther down the string the color change travels, the more saturated the tampon is.

It will be appreciated the contrast between the indicator threads 30^{b} and non-indicator threads of FIG. 3 in certain embodiments, such as those relying on pH and thermochromic dye, can aid with visibility such that an indication is easier for a user to detect. Such contrast can also aid visually differentiate the product from others on the market. Under certain indicator embodiments a natural wax coating or the like may be applied to non-indicator threads to further isolate them from indicator threads and further effectuate contrast.

Time-Lapse Indicator: Time lapse embodiments of the present invention can be configured to provide an indication after a predetermined period of time though use of a thermochromic dye or pigment. The predetermined time may be (for example) eight hours based on a Food and Drug Administration (FDA) recommendation that women limit wear-time per tampon to no more than eight hours. A thermochromic dye is introduced into the length of a waterproofed tampon string. Exemplary natural thermochromic dye or pigment compositions include, for example: anthocyanidins. Such thermochromic dye compositions change color based on a time-sensitive exposure to the user's body heat.

Operation of an exemplary time-lapse indicator occurs as follows. Prior to use the indicator threads of the removal string contain the thermochromic dye set to a specific color. While in use, the thermochromic dye in the indicator threads gradually changes color as it is exposed to body heat through skin contact. An elapsed time of approximately 6-8 hours results in an observable color change. The waterproof coating may act as a heat insulator, and the thickness of the waterproofing may be calibrated to control the reaction time of the thermochromic dye.

Sensory Indicator: In another embodiment, a sensory indicator is comprised of a natural material such as sorbitol crystals 30c, which creates a cooling sensation when exposed to moisture from menstrual fluid absorbed by the tampon. The indicator is contained between the inner layer of the pledget and protected from premature exposure to moisture by means of a permeable waterproofed layer either located on the outside of the pledget or as an inner layer within the pledget.

Operation of an exemplary sensory indicator occurs as follows. Prior to use the pledget and crystals are dry at 0% saturation level. While in use the pledget 10 increases in saturation as it is exposed to menstrual fluid. At approximately 60-80% saturation level, the moisture in the pledget reaches the sorbitol crystals, and the activated sorbitol crystals produce a cooling sensation noticeable to the user.

In one embodiment, the sorbitol crystals are sandwiched between two sheets of natural material such as cotton, to contain the crystals. A waterproofed layer such as natural wax (bees wax, soy wax, etc.) or bioplastic (made from corn, potatoes, rice, palm fiber, wood cellulose, wheat fiber, etc.) is applied to one of the sheets of cotton. The cotton-sorbitol assembly is layered within the tampon pledget such that as the tampon is formed, the waterproofed layer of the cotton-sorbitol assembly is facing towards the outer surface of the pledget. This provides immediate protection of the sorbitol crystals from moisture in the vaginal environment, while leaving the inner surface of the cotton-sorbitol assembly in communication with the interior of the pledget. This allows for menstrual fluid to activate the sorbitol crystals, but not until the pledget is substantially saturated. Placement of the cotton-sorbitol assembly at different locations along the axial length of the tampon pledget may contribute control of the indicator activation, because the tampon is exposed to the majority of menstrual fluid at the cervix,—essentially saturating the tampon from the leading end first.

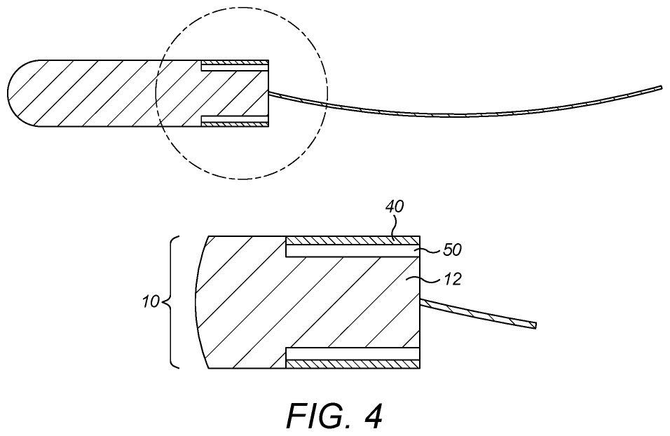

In accordance with FIG. 4, the pledget 10 may be comprised of multiple layers such that a sensory indicator 50 is located underneath the surface of the pledget 10 rather than within the absorbent core 12. In one embodiment, the pledget 10 has a waterproofed outer layer 40, followed by a sensory indicator 50 and the absorbent core 12.

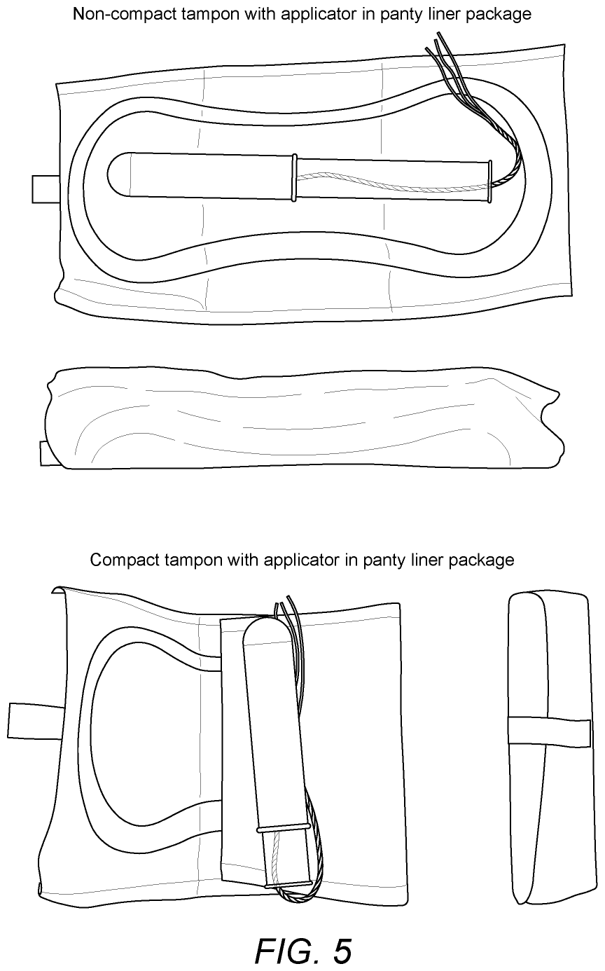

FIG. 5 depicts embodiments of a combined tampon and panty liner packaging configuration. As depicted, a panty shield is wrapped lengthwise or widthwise around a compact and non-compact tampon.

Any feature of any embodiment discussed herein may be combined with any feature of any other embodiment discussed or incorporated herein in some examples of implementation.

For example, sensory indicator embodiments in accordance with the discussion above can be adapted for use in a sanitary pad or liner, where the sorbitol crystals are disposed in a strip or section of the pad. This strip or section may be on top of or within the pad. In one embodiment, said strip or section has a waterproof containing layer on the top side, and a containing layer on the underside that communicates with the absorbent layers of the pad such that moisture from within the absorbent layers activate the sorbitol crystals to produce a cooling sensation indicator. Visual indicator embodiments can also be adapted for use in a color-changing strip or section of a pad or panty liner, where color change is triggered according to the three difference methods described above.

While various embodiments and examples have been presented, this was for the purpose of describing, but not limiting, the invention. Various modifications and enhancements will become apparent to those of ordinary skill in the art and are within the scope of the invention, which is defined by the appended claims.

1. A sanitary product comprising:
an absorbent pledget having a proximal end for placement near the cervical bone and a distal end opposite said proximal end; and a removal string being connected to said absorbent pledget, and an indicator coupled to or associated with the absorbent pledget and in contact with said removal string at said distal end, said indicator being visually perceivable on the waterproofed removal string when contacted by menstrual fluid.

2. The product of claim 1 wherein the indicator comprises a water-soluble dye that runs through the length of the waterproofed tampon string.

3. The product of claim 1 wherein the indicator comprises a thermochromic dye that runs through the length of the waterproofed tampon string.

4. The product of claim 1 wherein the indicator comprises a potential of hydrogen solution that runs through the length of the waterproofed tampon string.

5. The product of claim 4 wherein the saturation-limit indicator is adapted to react after being exposed to a pH change of approximately 4.0 to approximately 7.0.

6. The product of claim 1 wherein the removal string includes an outer surface coated with beeswax, soy wax, cottonseed wax, palm wax, other natural wax or any combination thereof.

7. A sanitary product comprising:
an absorbent pledget having a proximal end for placement near the cervix and a distal end opposite said proximal end; and a removal string being connected to said absorbent pledget, and an indicator coupled to or associated with the absorbent pledget and in contact with said removal string at said distal end, said indicator being sensually perceivable to the user when contacted by menstrual fluid.

8. The product of claim 7 wherein the indicator comprises a sensory material at least partially surrounded by, embedded in, or covered by the pledget and a waterproofed layer that protects the sensory material and prevents its release until after a predetermined saturation point has been reached.

9. The product of claim 8 wherein the indicator is comprised of Sorbitol crystals.

10. The product of claim 9 wherein the waterproofed layer is located on the outside of the pledget and is comprised of beeswax, soy wax, cottonseed wax, palm wax, other natural wax, bioplastic or any combination thereof.

11. The product of claim 9 wherein the waterproofed layer is located on the inside of the pledget and is comprised of beeswax, soy wax, cottonseed wax, palm wax, other natural wax, bioplastic or any combination thereof.
